(19) United States
(12) Patent Application Publication
(10) Pub. No.: US 2006/0161067 A1
(43) Pub. Date: Jul. 20, 2006

(54) Complexity scores for electrocardiography reading sessions

(75) Inventor: Jonathan L. Elion, Wakefield, RI (US)

Correspondence Address: HOUSTON ELISEEVA, 4 MILITIA DRIVE, SUITE 4, LEXINGTON, MA, 02421, US

(73) Assignee: Heartlab, Inc., Westerly, RI (US)

(21) Appl. No.: 11/335,870

(22) Filed: Jan. 18, 2006

Related U.S. Application Data
(60) Provisional application No. 60/644,876, filed on Jan. 18, 2005.

(51) Int. Cl. A61B 5/04

(52) U.S. Cl. 600/509

(57) ABSTRACT

A system allows for the prioritization of ECGs. This can be performed by the ECG management system and/or at the instruction of the cardiologist or other reader. In a current implementation, the system will allow for the sorting of the ECGs so that the more complex interpretations are presented first, when the cardiologist or other reader is not suffering from fatigue, saving the simpler readings for later in the session as fatigue might begins to become a factor.

### RELATED APPLICATIONS

This application claims the benefit under 35 USC 119(e) of U.S. Provisional Application No. 60/644,876, filed on Jan. 18, 2005, which is incorporated herein by reference in its entirety.

### BACKGROUND OF THE INVENTION

Electrocardiography is a technology for the detection and diagnosis of cardiac conditions. An electrocardiograph is a medical device capable of recording the potential differences generated by the electrical activity of the heart. An electrocardiogram (ECG or EKG) is produced by the electrocardiograph. It typically comprises the ECG wave data that describes the heart's electrical activity as a function of time.

The heart's electrical activity is detected by sensing electrical potentials via a series of electrode leads that are placed on the patient at defined locations on the patient's chest and limbs. Systems with ten (10) separate ECG leads and digital data capture/storage are typical. During electrocardiography, the detected electrical potentials are recorded and graphed as ECG wave data that characterize the depolarization and repolarization of the cardiac muscle.

ECG interpretation is performed by analyzing the various cardiac electrical events presented in the ECG wave data. Generally, the ECG wave data comprise a P wave, which indicates atrial depolarization, a QRS complex, which represents ventricular depolarization, and a T-wave representing ventricular repolarization.

State-of-the-art ECG systems provide for the machine interpretation of the ECG data. These systems are designed to measure features of the ECG wave data from the patient. The various features of portions of the ECG, such as intervals, segments and complexes, including their amplitude, direction, and duration of the waves and their morphological aspects, are measured. Then all of the feature information is analyzed together. From this feature information, these systems are able to generate machine ECG interpretations diagnosing normal and abnormal cardiac rhythms and conduction patterns. These interpretations are often used by the physician/cardiologist as the basis of an ECG report for a given patient.

### SUMMARY OF THE INVENTION

The standard clinical practice in most hospitals in the United States and elsewhere is for ECGs to be collected by technicians in the ECG department and presented to the responsible cardiologists to be interpreted. These cardiologists are often tasked with reviewing large numbers of ECGs from many different patients. But to ease this task, it is common that the ECGs will have already been read by a computer algorithm, and the computer's interpretation (a list of interpretive statements) will only need to be reviewed (“over-read”) by the cardiologist and any necessary changes noted.

In this common model of “batch reading,” the cardiologist is often confronted with over-reading a large number of electrocardiograms in one sitting. And, the cardiologist will encounter some degree of mental fatigue after reading for an extended sitting.

In conventional management systems, ECGs are presented for reading based on the patient name or based on the time that the ECGs were recorded. The ECG management system is not able to sort the ECGs in a way that is useful to the cardiologists or facilitate their work.

The present invention is directed to a system that allows for the prioritization of ECGs. This can be performed by the ECG management system and/or at the instruction of the cardiologist or other reader. In a current implementation, the system will allow for the sorting of the ECGs so that the more complex interpretations are presented first, when the reader is not suffering from fatigue, saving the simpler readings for later in the session as fatigue might begin to become a factor.

There are a number of potential ways of charactering the complexity of reading ECG data for a given patient. ECGs for a patient are examined and read as a group since the patient often has more than one ECG taken between the last reading session and the current one. In contrast, the simplest over-reading situation is the one where there is only one ECG to read for the patient. The more ECGs that have accumulated for a patient and that need to be read, the more complex the reading task becomes, since as ECGs have to be compared to each other, and this comparison is time consuming. Complexity also increases in direct proportion to the number of interpretive statements on each machine-generated ECG interpretation. Finally, certain diagnoses require more careful review than others do, and these diagnoses can be scored based on the differences in difficulty.

In general, according to one aspect, the invention features a method for presenting electrocardiogram (ECG) data to a reader, such as a cardiologist. The method comprises scoring ECG data from different patients based on a sorting criteria and then sorting the ECG data from the different patients. A reader then reviews the ECG data from the different patients in the order determined by the sorting.

In the typical application, this reader generates the ECG reports for the different patients.

The step of scoring the ECG data comprises comparing the ECG data from the different patients with respect to the sorting criteria. Often and in the preferred embodiment, the sorting criteria is a metric characterizing a complexity of the ECG data. One such metric is the number of previous ECGs that exist for the different patients. Alternatively, or in addition, machine-generated interpretations for the ECG data for the different patients can be compared to a list of diagnoses representing the sorting criteria. For example, more difficult diagnoses can be given a higher score.

In general, according to another aspect, the invention features a system for presenting electrocardiogram data to a reader. This system comprises a host system for scoring ECG data from different patients based on a sorting criteria and then sorting the ECG data from the different patients. A workstation is also provided that enables a reader to review the ECG data from the different patients in an order determined by the sorting.

In general, according to still another aspect, the invention features a computer software product for ECG data presentation. This product comprises a computer-readable medium in which program instructions are stored. These instructions, when read by a computer, cause the computer to score ECG data from different patients based on a sorting criteria and then sort the ECG data to be over-read by a reader from different patients, based on the sorting criteria. It also enables the reader to review the ECG data from the different patients in the order determined by the sorting.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram illustrating the electrocardiogram (ECG) workflow in a typical hospital;

FIG. 2 is a flow diagram illustrating the machine interpretation process in a conventional ECG device or host-based interpretation system;

FIG. 3 shows prototypical ECG wave data illustrating the various portions of the wave;

FIG. 4 shows a conventional interface in an ECG report editing system;

FIG. 5 shows a series of text statements as would be generated by machine interpretation for an exemplary ECG report as is conventional; and

FIG. 6 is a flow diagram illustrating the process for ECG scoring and complexity sorting according to the present invention.

### DETAILED DESCRIPTION OF THE PREFERRED EMBODIMENTS

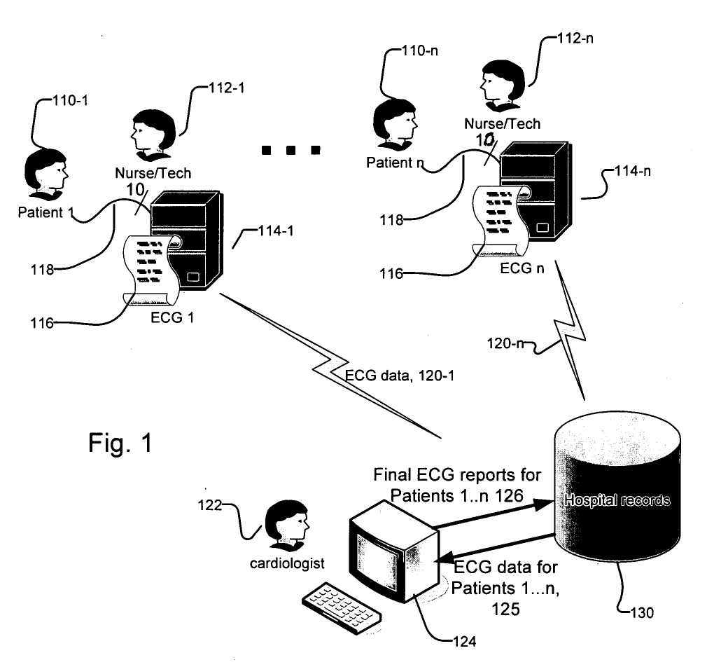

FIG. 1 illustrates the electrocardiogram (ECG) workflow in a typical hospital. A nurse or ECG technician 112-1 interacts with the patient 1 110-1 to acquire the ECG data. In many modem systems, the ECG machine 114-1 is an ECG cart that is moved throughout the hospital between patient, examining, and operating rooms.

In operation, the ten (10) leads 118 of the ECG device 114-1 are placed on the limbs and torso of the patient 110-1. Then, a printout of the ECG wave data 116 is generated at the cart. Also, ECG data 120-1 including the wave data using 12 combinations of the leads that have been placed on the patient and possibly a machine-generated ECG interpretation are generated and digitally stored in the ECG cart 114-1 and/or transmitted to a central hospital records data storage and host system 130.

In parallel, other nurses/technicians 112-n are taking ECGs of other patients 110-n such as patient n. All of the ECG data records 120-n are similarly sent back to the records database and host system 130. In modem hospitals, specifically, this is a central depository database of hospital records. Here the ECG data from all of the patients is accumulated.

The present invention generally applies to host based interpretation and editing systems. In these systems, a cardiologist 122 accesses the ECG data 125 from the records database 130 usually via a workstation 124. The hospital records and host system 130 will store preliminary ECG data, generate and store machine interpretations of the ECG data, and store the subsequent final reports 126 that are the product of the editing process by the cardiologist 122 at the workstation 124. The final reports will then be entered into the patients' records.

The workstation 124 is provided with standard software for accessing and editing the ECG data, machine-generated interpretations and reports from host system 130, and generating the final cardiologist-reviewed ECG reports. In the preferred implementation, the database and host system 130 or workstation 124 also has a host-based interpretation system that enables it to generate its own machine-generated interpretation using the ECG data 120 from the cart 114, for example, even when a cart-generated interpretation was made.

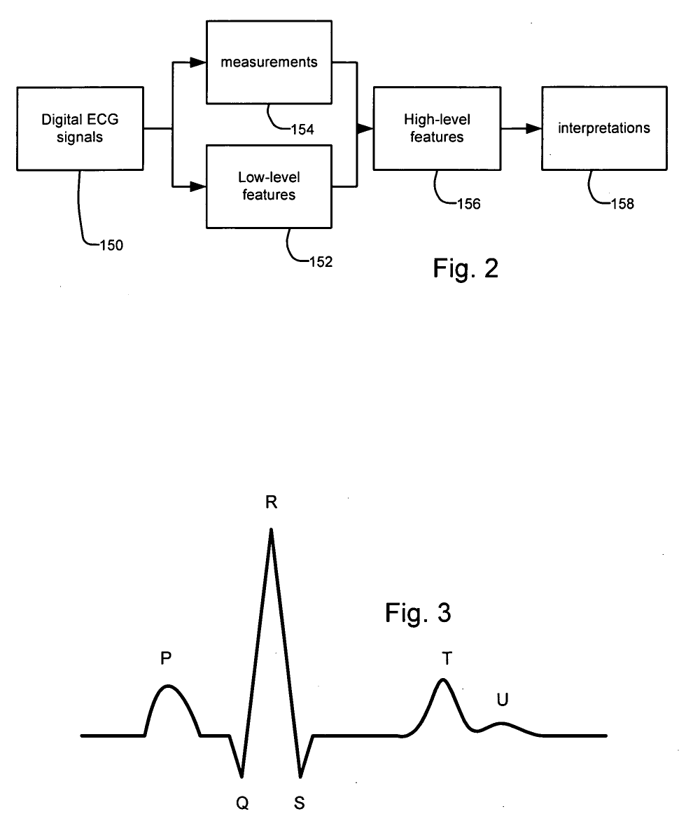

FIG. 2 illustrates the general process by which these machine interpretations are generated. Commonly, they are performed in the cart and/or in host-based interpretation systems. In either case, the raw ECG wave data are machine interpreted for the cardiologist or other reader.

Specifically, the digital ECG signals or wave data 150 are acquired in step 150 and stored such as by the ECG cart. Measurements of portions of this ECG wave data are made in step 154 and low-level features 152 are typical identified in the wave data at the host system 130. This information is then combined in step 156 where high-level features are determined. Based on these calculated features, the final machine interpretation is generated in step 158.

The features typically relate to the length and amplitude of the various components of a selected ECG wave from one typical cardiac cycle out of the usually very long wave data set that the machine acquires. In other cases, an average ECG wave is calculated from a series of waves to form the basis of the interpretation.

FIG. 3 illustrates a prototypical ECG wave. It generally comprises a P wave, a QRS wave complex, a T-wave, and a U wave. The features that the typical system uses can be dependent on specific characteristics of that system but will include intervals, segments and complexes, including amplitude, direction, and duration of the waves and their morphological aspects.

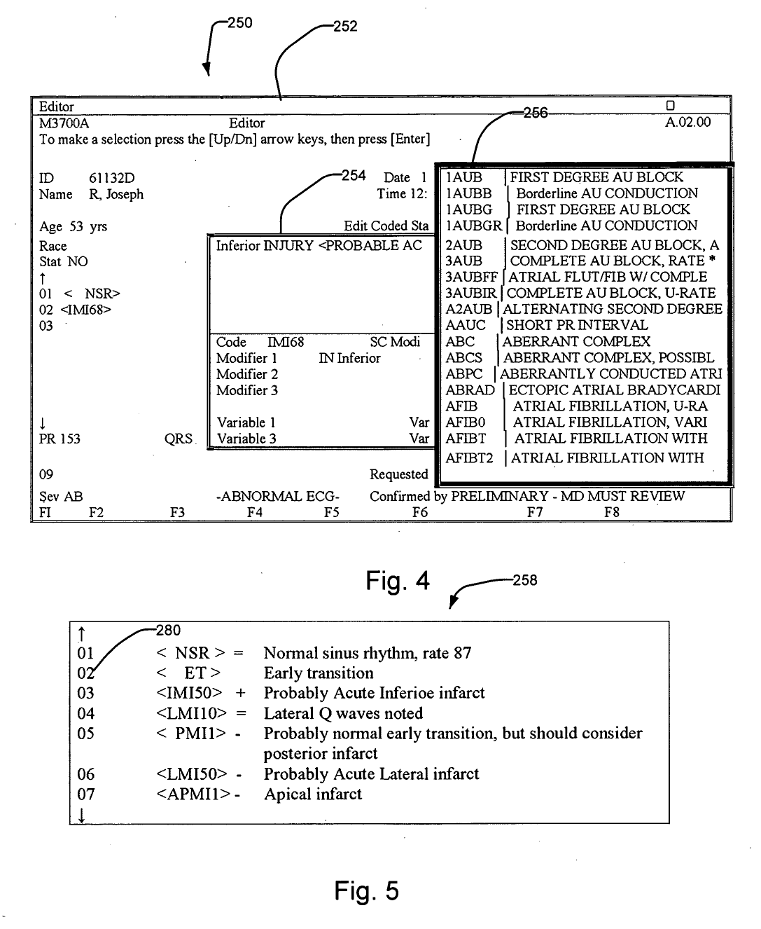

FIG. 4 illustrates a typical interface 250 for an ECG report editor running on workstation 124. In the specific example, it displays a window 252 that provides general information on the patient “R, Joseph.” It has another window 254 that provides a workspace for creating the final ECG report. Typically, these ECG reports are a set of specific codes, displayed in window 256 that correspond to different conditions.

FIG. 5 illustrates an exemplary draft report 258 as generated by a machine interpretation. It comprises a series of lines that correspond to different conditions. Typically, they are ordered in their relative importance. The physician, at the workstation, will review the specific ECG wave data and revise the draft report generated from the machine interpretation. These series of statements 01-07 (280), providing specific diagnoses, will then be edited in order to generate the final report that is stored in the patient database 130.

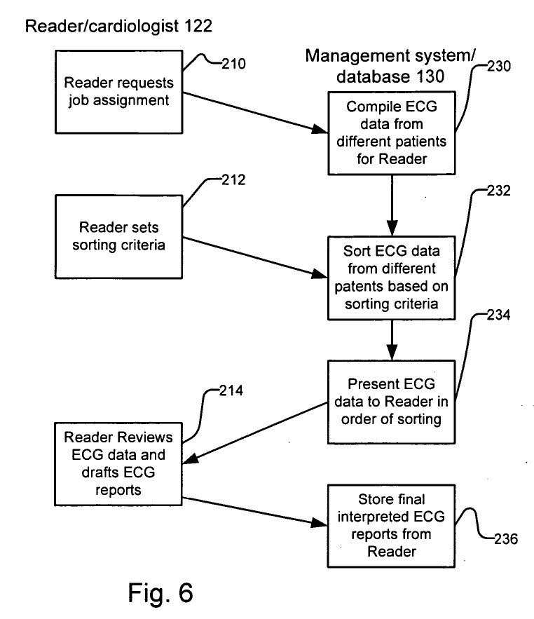

FIG. 6 illustrates a method for presenting electrocardiogram (ECG) data to a reader. Specifically, as in the past, the digital ECG data including the interpretations, typically from the ECG cart, are received at the database and host system 130 for many patients. Then the cardiologists/readers will request a job assignment in step 210.

The process of requesting the job assignment can be relatively simple or complex depending on the type of system used. In some systems, the reader requests a job assignment simply by accessing a file that has the batch of ECGs that are pending be read. In other examples, the database and host system 130 compiles the batches of ECGs from the different patients and then distributes them among the cardiologists/readers that are working on batch over-reads.

Typically, this distribution of the patients among the cardiologists is based upon which individuals are patients of the various cardiologists. In other examples, the system will assign the ECGs to be read among the various cardiologists to achieve an even workload distribution. In any case, the ECG data for the different patients are then compiled by the database system 130 or by the workstation 124 accessing the pending jobs based on the cardiologist request in step 230.

In step 212, the cardiologist or other reader sets the sorting criteria according to the invention. In the current embodiment, the reader sets sorting criteria that are based on the complexity of the ECG data to be read. Specifically, the reader 122 will often request that the batch of ECG data from the different patients be sorted in decreasing complexity in terms of the process of reading the ECG data from the different patients. In other examples, the reader may present sorting criteria that requests ECG data to be sorted based on increasing complexity.

Then in step 232, the database or management system sorts the ECG data from the different patients based on the sorting criteria. In one example, where the sorting criteria are based on complexity, the station 124 or database hosting system 130 calculates a complexity score for the ECG data from each of the patients. This complexity score is a metric characterizing the complexity of task of reading the ECG data and generating the report for that patient.

In the preferred embodiment, there are a number of ways of characterizing the complexity of the ECG data for a given patient. In one example, the number of previous ECGs that exist for each of the different patients is used as a metric. Typically, the complexity of reading ECG data increases as the number of other ECG data sets from that patient increases since more ECG data sets must be compared to each other in order to determine how the patient's health is changing. In other examples, the complexity of the ECG report is characterized based on the number of machine-generated interpretive statements present in the ECG data. In still other examples, each of the different potential diagnoses for all of the patients is given a score by a reviewing physician, based on the assessment of the complexity of the different diagnoses. Then, the ECG data for the different patients are sorted based upon that complexity list, and specifically the machine-generated interpretation of the ECG data.

Then in step 234, the ECG data of the patients is presented to the reader in the order generated from the sorting in step 234.

The reader 122 then reviews the ECG data from the management system database 130 and drafts the ECG reports for the different patients in step 214. The final interpreted ECG reports from the reader are then stored in the database management system 130 in step 236.

According to another embodiment, at the time of receipt at the management database host system 130, a complexity scores is assigned to the ECG data, usually based on the result of the machine-generated interpretation. These complexity scores are made available to the cardiologists/readers 122 allowing the readers to thereby sort their reports during a batch reading, for example, based on this complexity score.

In other examples, the management systems database 130 uses the complexity scores to affect load distribution across a number of cardiologists or other readers working at a hospital, for example. This will allow the system, in some examples, to assign the more difficult reading tasks to the more experienced cardiologists. In other examples, the management system/database 130 compares the complexity scores of the ECG data and then creates batches of ECG data to be read by the cardiologist such that all cardiologists have a similar mix of difficult and easy ECG data over-reading tasks.

The following illustrates specific approaches for generating the complexity score.

1. (Number of ECGs×10)+average number of interpretive statements per ECG—this formula takes into account the number of ECGs to be read for the patient and the complexity of the expected diagnoses.

2. Sum of diagnostic complexity scores—each interpretive statement is assigned a complexity score between 0 to 4, easy to hard respectively. The score for a given ECG is equal to the sum of the complexity scores of each interpretive statement that has been provided by the computer analysis of the machine-generated interpretation; the complexity score for the patient is equal to the sum of the complexity scores for each of the ECGs to be over-read.

Example: The ECG reading workstation 124 presents a list of ECGs to be reviewed to the over-reading cardiologist or other user 122. The order in which these are presented is based on the ECG reading complexity score, presented in decreasing complexity order in one embodiment. By simply requesting “Next Patient,” the patient with the highest complexity score is selected to be reviewed next. This assures that the more difficult interpretive tasks are presented at the beginning of the over-reading session while the cardiologist is still fresh, while the simpler interpretive tasks are saved for the end of the reading session when fatigue may be a significant factor.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for presenting electrocardiogram (ECG) data to a reader, the method comprising:
scoring ECG data from different patients based on sorting criteria;
sorting the ECG data from the different patients based on the sorting criteria; and
a reader reviewing the ECG data from the different patients in an order determined by the sorting.

2. A method as claimed in claim 1, further comprising the reader generating ECG reports for the different patients from the ECG data.

3. A method as claimed in claim 1, wherein the step of scoring the ECG data comprises comparing the ECG data from the different patients with respect to the sorting criteria.

4. A method as claimed in claim 1, wherein the sorting criteria includes a metric characterizing a complexity of ECG data.

5. A method as claimed in claim 1, wherein the sorting criteria includes a metric characterizing a number of previous ECGs that exist for each of the different patients.

6. A method as claimed in claim 1, wherein the step of scoring the ECG data comprises comparing machine-generated interpretations in the ECG data to list of diagnoses representing the sorting criteria.

7. A method as claimed in claim 6, further comprising scoring the list of diagnoses based on a relative complexity of each diagnosis.

8. A method as claimed in claim 1, wherein the sorting criteria is to review more complex ECG data first.

9. A method as claimed in claim 1, further comprising compiling the ECG data from the different patients to be read by a reader requesting a job assignment.

10. A method for presenting electrocardiogram (ECG) data to readers, the method comprising:
compiling ECG data from different patients for presentation to a reader for generation of ECG reports for the different patients;
analyzing the ECG data from the different patients and sorting the ECG data based on a sorting criteria; and
presenting the ECG data from the different patients in an order determined by the sorting.

11. A system for presenting electrocardiogram (ECG) data to a reader, the system comprising:
a host system for scoring ECG data from different patients based on sorting criteria and sorting the ECG data from the different patients based on the sorting criteria; and
a workstation enabling a reader to review the ECG data from the different patients in an order determined by the sorting.

12. A system as claimed in claim 11, further comprising the reader generating ECG reports on the workstation for the different patients from the ECG data.

13. A system as claimed in claim 11, wherein the host system scores the ECG data by comparing the ECG data from the different patients with respect to the sorting criteria.

14. A system as claimed in claim 11, wherein the sorting criteria includes a metric characterizing a complexity of ECG data which is determined by the host system.

15. A system as claimed in claim 11, wherein the sorting criteria includes a metric characterizing a number of previous ECGs that exist for each of the different patients.

16. A system as claimed in claim 11, wherein the host system scores the ECG data by comparing machine-generated interpretations in the ECG data to a list of diagnoses representing the sorting criteria.

17. A system as claimed in claim 16, further comprising scoring the list of diagnoses based on a relative complexity of each diagnosis.

18. A system as claimed in claim 11, wherein the sorting criteria is to review more complex ECG data first.

19. A system as claimed in claim 11, wherein the host system compiles the ECG data from the different patients to be read by the reader requesting a job assignment.

20. A computer software product for ECG data presentation, the product comprising a computer-readable medium in which program instructions are stored, which instructions, when read by a computer, cause the computer to score ECG data from different patients based on sorting criteria, sort the ECG data to be over-read by a reader from the different patients based on the sorting criteria, and enable the reader to review the ECG data from the different patients in an order determined by the sorting.

21. A product as claimed in claim 20, wherein the instruction further cause the computer to score the ECG data based on a complexity of ECG data.

22. A product as claimed in claim 20, wherein the instructions further cause the computer to score the ECG data based on a number of previous ECGs that exist for each of the different patients.

23. A method as claimed in claim 20, wherein the instructions further cause the computer to compare machine-generated interpretations in the ECG data to a list of diagnoses representing the sorting criteria.
